(19) United States
(12) Patent Application Publication
(10) Pub. No.: US 2022/0104778 A1
(43) Pub. Date: Apr. 7, 2022

(54) Systems and Methods for a Personal Diagnostic Device

(71) Applicant: Keyshare Innovation Group LLC, Seal Beach, CA (US)

(72) Inventor: Robert Safari, Seal Beach, CA (US)

(73) Assignee: Keyshare Innovation Group LLC, Seal Beach, CA (US)

(21) Appl. No.: 17/450,114

(22) Filed: Oct. 6, 2021

Related U.S. Application Data
(60) Provisional application No. 63/088,742, filed on Oct. 7, 2020.
(60) Provisional application No. 63/088,747, filed on Oct. 7, 2020.
(60) Provisional application No. 63/088,748, filed on Oct. 7, 2020.

(51) Int. Cl. A61B 5/00; G16H 80/00; G16H 40/63; G16H 10/60; A61B 5/0205; A61B 5/024; A61B 5/1455; A61B 7/02; A61L 2/10

(52) U.S. Cl. CPC A61B 5/744; A61L 2202/24; G16H 40/63; G16H 10/60; A61B 5/748; A61B 5/02055; A61B 5/02444; A61B 5/14552; A61B 7/02; A61B 5/7405; A61B 5/0002; A61B 5/746; A61B 5/7445; A61L 2/10; A61B 2562/0271; A61L 2202/11; G16H 80/00

(57) ABSTRACT

The present invent is directed to a diagnostic platform that can be used to provide efficient and safe healthcare. The diagnostic platform has a database and back-end server for storing and processing healthcare information, a display for conveying information to a user, and a user interface for interacting with a user that includes an avatar that can assist the user. A diagnostic device including a temperature sensor, a heart rate sensor, a pulse oximeter sensor; and a stethoscope may be used to obtain healthcare information from the user. A system of electronic keys and electronic key readers may be integrated into the diagnostic platform to provide contract tracing functionality.

### CROSS-REFERENCE TO RELATED APPLICATIONS

The present application claims the benefit of U.S. Provisional Application No. 63/088,742 filed Oct. 7, 2020, by Robert Safari and titled “Systems and Methods for a Personal Diagnostic Platform”, U.S. Provisional Application No. 63/088,747 filed Oct. 7, 2020, by Robert Safari and titled “Systems and Methods for a Personal Diagnostic Device”, and U.S. Provisional Application No. 63/088,748 filed Oct. 7, 2020, by Robert Safari and titled “Systems and Methods for a Personal Diagnostic Platform with Contact Tracing”, all of which are included by reference herein and for which benefit of the priority date is hereby claimed.

### FEDERALLY SPONSORED RESEARCH

Not applicable.

### SEQUENCE LISTING OR PROGRAM

Not applicable.

### FIELD OF INVENTION

The embodiments described herein are related to a personal diagnostic platform, and in particular to systems and methods that monitor health conditions, track location and contacts, and provide actionable data to healthcare and other professionals.

### BACKGROUND OF THE INVENTION

The recent pandemic has upended many industries, and as these industries try to come back online a gating issue is how to keep their people and their employees from getting COVID-19 as they come back. For example, the cruise industry was greatly affected by the pandemic. According to a report published by KPMG in July of 2020, the cruise industry was the fastest growing sector of the travel industry. But as the pandemic broke out, thousands of passengers were kept at see as countries closed their ports to foreign cruise ships fearing they would be a source of spread of the virus.

As noted that the current COVID-19 environment has created a high degree of concern amongst the public surrounding the maintenance of health and safety onboard cruise ships. Ships will now require robust screening and monitoring protocols, implementation of comprehensive sanitation practices with regular inspections, expanded onboard medical facilities and increased medical staff. Also, cruise liners will be expected to work more closely with public health authorities worldwide and the Cruise Lines International Association to enforce health requirements.” But the question is how to effectively and efficiently comply with the protocols in a cost effective and unobtrusive manner?

The pandemic has resulted in a large increase in the use of exploitation of telemedicine and remote monitoring devices. Data from GlobalData's latest research report, ‘Use of Telemedicine During the COVID-19 Pandemic in the US’, shows the analysis of mobile app download data from PrioriData. It found that most leading providers of patient and physician platforms experienced an increase in downloads from March, with many seeing more activity in Q2 2020 compared to all of 2019. These devices and telehealth platforms allow for the delivery of healthcare while decreasing the risk of spread of the virus to patients and healthcare professionals.

But players in industries such as the cruise industry still need tools that allow them to implement protocols on their ships or in their facilities and campuses to maintain health and safety and allow employees, visitors, customers, etc. to return.

### SUMMARY OF THE INVENTION

Systems and methods for personal diagnostic platform are described herein.

According to one aspect, a diagnostic platform, comprising: a database; an in-room unit, comprising: a display, and a user interface displayed via the display, the user interface comprising: an avatar capable of two-way verbal communication that can provide information and help navigate through screens that comprise the user interface, and a plurality of screens configured to provide information and allow or prompt the input if health information; and a back-end server configured to: receive the health information, make the health information available to healthcare personnel, and store the healthcare information. Avatars are generic third-party components. Avatars can also be motion-captured people, and includes real-time facial expressions. This could include live motion-captured avatars for real-time interaction with patients. Avatars can also be represented in holographic form in hologram devices. Avatars can also be represented as life-sized agents in holographic form in large hologram devices.

In one embodiment of the present invention, the diagnostic platform comprises a back-end server and an interface that can include an avatar. The diagnostic platform can present an avatar in a user interface. The avatar can act as a nurse, mental health specialist, and crisis prevention specialist. The avatar and associated AI software can be installed into a platform or a device. The diagnostic device does an interactive capture of telemedicine data and has the capability to check various datapoints related to health biometrics. The diagnostic platform can run on, or run in conjunction with, hologram devices, smart mirrors, business kiosks, and health check stations. The diagnostic platform can be integrated with cruise ship and hotel solutions, business offices, lobby check-in platforms, and in-room concierge platforms.

The diagnostic device can run data capture software locally, or can transmit in real time to data capture unit. The software can be installed on a CPU, and it can be connected to any display interface, for example to a TV through a HDMI connection, in which case the TV can be used as the interface. In one embodiment of the present invention, the software is Android based and is capable of capturing data from the diagnostic device through Bluetooth and streaming it live into the interface.

In one embodiment of the present invention, the captured telemedicine data is stored locally on the CPU. The data can then be directed to an external server within a business, or a HIPPA compliant server if required. Alternatively, the data can be streamed without retention. The software provides a GUI interface that can be layered into any service provider's existing application.

In one embodiment of the present invention, the software is a chatbot installed on a chip in a custom designed PCB board. The chatbot is trained to interact with a user by layering the data back and forth. The firmware of the diagnostic device can be integrated with software on the Android CPU that is used as the primary motherboard and storage.

During the telemedicine interaction, a health care provider can prescribe medication in real time. That interaction becomes another layer in the system. That information can be retained and subsequently provide the user with information on delivery dates and dosage reminders. The back-end server can negotiate fulfillment of the prescription from drug providers.

According to one aspect of the present invention, the diagnostic platform includes a plurality of readers, each configured to communicate with a plurality of electronic keys and to communicate information related to the location of the electronic keys and proximity to other electronic keys to the back-end-server so that the data can be used for contact tracing if needed.

According to one aspect, a personal diagnostic device, comprising: a handle, a contactless temperature sensor, a heart rate sensor, a pulse oximetry sensor, and a stethoscope.

These and other features, aspects, and embodiments are described below in the section entitled “Detailed Description.”

### BRIEF DESCRIPTION OF THE DRAWINGS

A complete understanding of the present invention may be obtained by reference to the accompanying drawings, when considered in conjunction with the subsequent, detailed description, in which:

FIG. 1 illustrates an example infrastructure in which one or more of the disclosed processes may be implemented, according to an embodiment.

FIG. 2 is a block diagram illustrating an example wired or wireless system that may be used in connection with various embodiments described herein.

FIG. 3 is a screen shot illustrating a user interface for the personal diagnostic platform according to one embodiment.

FIG. 4 is a screenshot illustrating a user interface that can be included in the platform of FIG. 3.

FIG. 5 is a flow chart illustrating an example of at least a portion of a series of questions that can be asked via the user interface in order to illicit information and data to use by the telemedicine module, or a healthcare professional.

FIG. 6 illustrates an example user interface for medical staff that can be included in the platform of FIG.

FIG. 7 is a block diagram of the sensor components of a diagnostic device.

FIG. 8 is a block diagram of input/output components of a diagnostic device.

FIG. 9 is a front oblique diagram illustrating another example embodiment of a diagnostic device.

FIG. 10 is a back oblique diagram illustrating another example embodiment of a diagnostic device.

FIG. 11 is a bottom oblique diagram illustrating another example embodiment of a diagnostic device.

FIG. 12 is a front oblique exploded diagram illustrating another example embodiment of a diagnostic device.

FIG. 13 is a flow chart illustrating the contact tracing process.

### DETAILED DESCRIPTION

Before the invention is described in further detail, it is to be understood that the invention is not limited to the particular embodiments described, as such may, of course, vary. It is also to be understood that the terminology used herein is for the purpose of describing particular embodiments only, and not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed with the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, a limited number of the exemplary methods and materials are described herein.

It must be noted that as used herein and in the appended claims, the singular forms “a”, “an”, and “the” include plural referents unless the context clearly dictates otherwise.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, if dates of publication are provided, they may be different from the actual publication dates and may need to be confirmed independently.

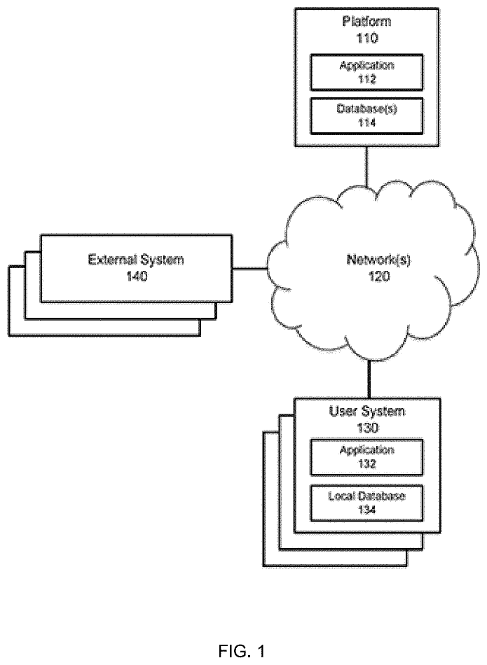

FIG. 1 illustrates an example infrastructure in which one or more of the disclosed processes may be implemented, according to an embodiment. The infrastructure may comprise a platform 110 (e.g., one or more servers) which hosts and/or executes one or more of the various functions, processes, methods, and/or software modules described herein. Platform 110 may comprise dedicated servers, or may instead comprise cloud instances, which utilize shared resources of one or more servers. These servers or cloud instances may be collocated and/or geographically distributed. Platform 110 may also comprise or be communicatively connected to a server application 112 and/or one or more databases 114. In addition, platform 110 may be communicatively connected to one or more user systems 130 via one or more networks 120. Platform 110 may also be communicatively connected to one or more external systems 140 (e.g., other platforms, websites, etc.) via one or more networks 120.

Network(s) 120 may comprise the Internet, and platform 110 may communicate with user system(s) 130 through the Internet using standard transmission protocols, such a s HyperText Transfer Protocol (HTTP), HTTP Secure (HTTPS), File Transfer Protocol (FTP), FTP Secure (FTPS), Secure Shell FTP (SFTP), and the like, as well as proprietary protocols. While platform 110 is illustrated as being connected to various systems through a single set of network(s) 120, it should be understood that platform 110 may be connected to the various systems via different sets of one or more networks. For example, platform 110 may be connected to a subset of user systems 130 and/or external systems 140 via the Internet, but may be connected to one or more other user systems 130 and/or external systems 140 via an intranet. Furthermore, while only a few user systems 130 and external systems 140, one server application 112, and one set of database(s) 114 are illustrated, it should be understood that the infrastructure may comprise any number of user systems, external systems, server applications, and databases.

User system(s) 130 may comprise any type or types of computing devices capable of wired and/or wireless communication, including without limitation, desktop computers, laptop computers, tablet computers, smart phones or other mobile phones, servers, game consoles, televisions, set-top boxes, electronic kiosks, point-of-sale terminals, Automated Teller Machines, and/or the like.

Platform 110 may comprise web servers which host one or more websites and/or web services. In embodiments in which a website is provided, the website may comprise a graphical user interface, including, for example, one or more screens (e.g., webpages) generated in HyperText Markup Language (HTML) or other language. Platform 110 transmits or serves one or more screens of the graphical user interface in response to requests from user system(s) 130. In some embodiments, these screens may be served in the form of a wizard, in which case two or more screens may be served in a sequential manner, and one or more of the sequential screens may depend on an interaction of the user or user system 130 with one or more preceding screens. The requests to platform 110 and the responses from platform 110, including the screens of the graphical user interface, may both be communicated through network(s) 120, which may include the Internet, using standard communication protocols (e.g., HTTP, HTTPS, etc.). These screens (e.g., webpages) may comprise a combination of content and elements, such as text, images, videos, animations, references (e.g., hyperlinks), frames, inputs (e.g., textboxes, text areas, checkboxes, radio buttons, drop-down menus, buttons, forms, etc.), scripts (e.g., JavaScript), and the like, including elements comprising or derived from data stored in one or more databases (e.g., database(s) 114) that are locally and/or remotely accessible to platform 110. Platform 110 may also respond to other requests from user system(s) 130.

Platform 110 may further comprise, be communicatively coupled with, or otherwise have access to one or more database(s) 114. For example, platform 110 may comprise one or more database servers which manage one or more databases 114. A user system 130 or server application 112 executing on platform 110 may submit data (e.g., user data, form data, etc.) to be stored in database(s) 114, and/or request access to data stored in database(s) 114. Any suitable database may be utilized, including without limitation MySQL™, Oracle™, IBM™ Microsoft SQL™, Access™, PostgreSQL™, and the like, including cloud-based databases and proprietary databases. Data may be sent to platform 110, for instance, using the well-known POST request supported by HTTP, via FTP, and/or the like. This data, as well as other requests, may be handled, for example, by server-side web technology, such as a servlet or other software module (e.g., comprised in server application 112), executed by platform 110.

In embodiments in which a web service is provided, platform 110 may receive requests from external system(s) 140, and provide responses in eXtensible Markup Language (XML), JavaScript Object Notation (JSON), and/or any other suitable or desired format. In such embodiments, platform 110 may provide an application programming interface (API) which defines the manner in which user system(s) 130 and/or external system(s) 140 may interact with the web service. Thus, user system(s) 130 and/or external system(s) 140 (which may themselves be servers), can define their own user interfaces, and rely on the web service to implement or otherwise provide the backend processes, methods, functionality, storage, and/or the like, described herein. For example, in such an embodiment, a client application 132 executing on one or more user system(s) 130 may interact with a server application 112 executing on platform 110 to execute one or more or a portion of one or more of the various functions, processes, methods, and/or software modules described herein. Client application 132 may be “thin,” in which case processing is primarily carried out server-side by server application 112 on platform 110. A basic example of a thin client application 132 is a browser application, which simply requests, receives, and renders webpages at user system(s) 130, while server application 112 on platform 110 is responsible for generating the webpages and managing database functions. Alternatively, the client application may be “thick,” in which case processing is primarily carried out client-side by user system(s) 130. It should be understood that client application 132 may perform an amount of processing, relative to server application 112 on platform 110, at any point along this spectrum between “thin” and “thick,” depending on the design goals of the particular implementation. In any case, the application described herein, which may wholly reside on either platform 110 (e.g., in which case server application 112 performs all processing) or user system(s) 130 (e.g., in which case client application 132 performs all processing) or be distributed between platform 110 and user system(s) 130 (e.g., in which case server application 112 and client application 132 both perform processing), can comprise one or more executable software modules that implement one or more of the processes, methods, or functions of the application described herein.

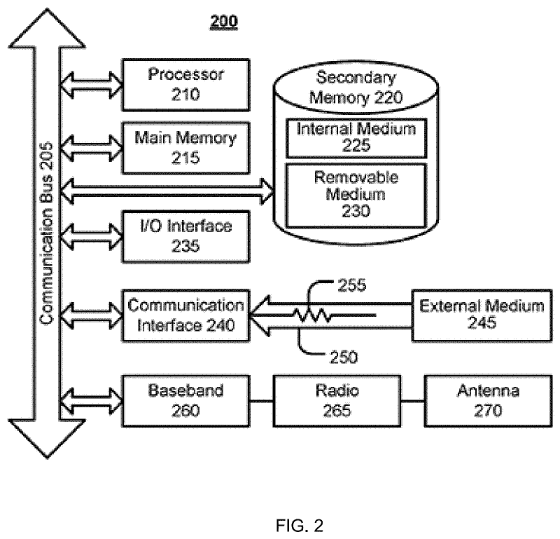

FIG. 2 is a block diagram illustrating an example wired or wireless system 200 that may be used in connection with various embodiments described herein. For example, system 200 may be used as or in conjunction with one or more of the functions, processes, or methods (e.g., to store and/or execute the application or one or more software modules of the application) described herein, and may represent components of platform 110, user system(s) 130, external system(s) 140, and/or other processing devices described herein. System 200 can be a server or any conventional personal computer, or any other processor-enabled device that is capable of wired or wireless data communication. Other computer systems and/or architectures may be also used, as will be clear to those skilled in the art.

System 200 preferably includes one or more processors, such as processor 210. Additional processors may be provided, such as an auxiliary processor to manage input/output, an auxiliary processor to perform floating-point mathematical operations, a special-purpose microprocessor having an architecture suitable for fast execution of signal-processing algorithms (e.g., digital-signal processor), a slave processor subordinate to the main processing system (e.g., back-end processor), an additional microprocessor or controller for dual or multiple processor systems, and/or a coprocessor. Such auxiliary processors may be discrete processors or may be integrated with processor 210. Examples of processors which may be used with system 200 include, without limitation, the Pentium® processor, Core i7® processor, and Xeon® processor, all of which are available from Intel Corporation of Santa Clara, Calif.

Processor 210 is preferably connected to a communication bus 205. Communication bus 205 may include a data channel for facilitating information transfer between storage and other peripheral components of system 200. Furthermore, communication bus 205 may provide a set of signals used for communication with processor 210, including a data bus, address bus, and/or control bus (not shown). Communication bus 205 may comprise any standard or non-standard bus architecture such as, for example, bus architectures compliant with industry standard architecture (ISA), extended industry standard architecture (EISA), Micro Channel Architecture (MCA), peripheral component interconnect (PCI) local bus, standards promulgated by the Institute of Electrical and Electronics Engineers (IEEE) including IEEE 488 general-purpose interface bus (GPIB), IEEE 696/S-100, and/or the like.

System 200 preferably includes a main memory 215 and may also include a secondary memory 220. Main memory 215 provides storage of instructions and data for programs executing on processor 210, such as one or more of the functions and/or modules discussed herein. It should be understood that programs stored in the memory and executed by processor 210 may be written and/or compiled according to any suitable language, including without limitation CIC++, Java, JavaScript, Perl, Visual Basic, .NET, and the like. Main memory 215 is typically semiconductor-based memory such as dynamic random access memory (DRAM) and/or static random access memory (SRAM). Other semiconductor-based memory types include, for example, synchronous dynamic random access memory (SDRAM), Rambus dynamic random access memory (RDRAM), ferroelectric random access memory (FRAM), and the like, including read only memory (ROM).

Secondary memory 220 may optionally include an internal medium 225 and/or a removable medium 230. Removable medium 230 is read from and/or written to in any well-known manner. Removable storage medium 230 may be, for example, a magnetic tape drive, a compact disc (CD) drive, a digital versatile disc (DVD) drive, other optical drive, a flash memory drive, and/or the like.

Secondary memory 220 is a non-transitory computer-readable medium having computer-executable code (e.g., disclosed software modules) and/or other data stored thereon. The computer software or data stored on secondary memory 220 is read into main memory 215 for execution by processor 210.

In alternative embodiments, secondary memory 220 may include other similar means for allowing computer programs or other data or instructions to be loaded into system 200. Such means may include, for example, a communication interface 240, which allows software and data to be transferred from external storage medium 245 to system 200. Examples of external storage medium 245 may include an external hard disk drive, an external optical drive, an external magneto-optical drive, and/or the like. Other examples of secondary memory 220 may include semiconductor-based memory, such as programmable read-only memory (PROM), erasable programmable read-only memory (EPROM), electrically erasable read-only memory (EEPROM), and flash memory (block-oriented memory similar to EEPROM).

As mentioned above, system 200 may include a communication interface 240. Communication interface 240 allows software and data to be transferred between system 200 and external devices (e.g. printers), networks, or other information sources. For example, computer software or executable code may be transferred to system 200 from a network server (e.g., platform 110) via communication interface 240. Examples of communication interface 240 include a built-in network adapter, network interface card (NIC), Personal Computer Memory Card International Association (PCMCIA) network card, card bus network adapter, wireless network adapter, Universal Serial Bus (USB) network adapter, modem, a wireless data card, a communications port, an infrared interface, an IEEE 1394 fire-wire, and any other device capable of interfacing system 200 with a network (e.g., network(s) 120) or another computing device. Communication interface 240 preferably implements industry-promulgated protocol standards, such as Ethernet IEEE 802 standards, Fiber Channel, digital subscriber line (DSL), asynchronous digital subscriber line (ADSL), frame relay, asynchronous transfer mode (ATM), integrated digital services network (ISDN), personal communications services (PCS), transmission control protocol/Internet protocol (TCP/IP), serial line Internet protocol/point to point protocol (SLIP/PPP), and so on, but may also implement customized or non-standard interface protocols as well.

Software and data transferred via communication interface 240 are generally in the form of electrical communication signals 255. These signals 255 may be provided to communication interface 240 via a communication channel 250. In an embodiment, communication channel 250 may be a wired or wireless network (e.g., network(s) 120), or any variety of other communication links. Communication channel 250 carries signals 255 and can be implemented using a variety of wired or wireless communication means including wire or cable, fiber optics, conventional phone line, cellular phone link, wireless data communication link, radio frequency (“RF”) link, or infrared link, just to name a few.

Computer-executable code (e.g., computer programs, such as the disclosed application, or software modules) is stored in main memory 215 and/or secondary memory 220. Computer programs can also be received via communication interface 240 and stored in main memory 215 and/or secondary memory 220. Such computer programs, when executed, enable system 200 to perform the various functions of the disclosed embodiments as described elsewhere herein.

In this description, the term “computer-readable medium” is used to refer to any non-transitory computer-readable storage media used to provide computer-executable code and/or other data to or within system 200. Examples of such media include main memory 215, secondary memory 220 (including internal memory 225, removable medium 230, and external storage medium 245), and any peripheral device communicatively coupled with communication interface 240 (including a network information server or other network device). These non-transitory computer-readable media are means for providing executable code, programming instructions, software, and/or other data to system 200.

In an embodiment that is implemented using software, the software may be stored on a computer-readable medium and loaded into system 200 by way of removable medium 230, I/O interface 235, or communication interface 240. In such an embodiment, the software is loaded into system 200 in the form of electrical communication signals 255. The software, when executed by processor 210, preferably causes processor 210 to perform one or more of the processes and functions described elsewhere herein.

In an embodiment, I/O interface 235 provides an interface between one or more components of system 200 and one or more input and/or output devices. Example input devices include, without limitation, sensors, keyboards, touch screens or other touch-sensitive devices, biometric sensing devices, computer mice, trackballs, pen-based pointing devices, and/or the like. Examples of output devices include, without limitation, other processing devices, cathode ray tubes (CRTs), plasma displays, light-emitting diode (LED) displays, liquid crystal displays (LCDs), printers, vacuum fluorescent displays (VFDs), surface-conduction electron-emitter displays (SEDs), field emission displays (FEDs), and/or the like. In some cases, an input and output device may be combined, such as in the case of a touch panel display (e.g., in a smartphone, tablet, or other mobile device).

System 200 may also include optional wireless communication components that facilitate wireless communication over a voice network and/or a data network (e.g., in the case of user system 130). The wireless communication components comprise an antenna system 270, a radio system 265, and a baseband system 260. In system 200, radio frequency (RF) signals are transmitted and received over the air by antenna system 270 under the management of radio system 265.

In an embodiment, antenna system 270 may comprise one or more antennae and one or more multiplexors (not shown) that perform a switching function to provide antenna system 270 with transmit and receive signal paths. In the receive path, received RF signals can be coupled from a multiplexor to a low noise amplifier (not shown) that amplifies the received RF signal and sends the amplified signal to radio system 265.

In an alternative embodiment, radio system 265 may comprise one or more radios that are configured to communicate over various frequencies. In an embodiment, radio system 265 may combine a demodulator (not shown) and modulator (not shown) in one integrated circuit (IC). The demodulator and modulator can also be separate components. In the incoming path, the demodulator strips away the RF carrier signal leaving a baseband receive audio signal, which is sent from radio system 265 to baseband system 260.

If the received signal contains audio information, then baseband system 260 decodes the signal and converts it to an analog signal. Then the signal is amplified and sent to a speaker. Baseband system 260 also receives analog audio signals from a microphone. These analog audio signals are converted to digital signals and encoded by baseband system 260. Baseband system 260 also encodes the digital signals for transmission and generates a baseband transmit audio signal that is routed to the modulator portion of radio system 265. The modulator mixes the baseband transmit audio signal with an RF carrier signal, generating an RF transmit signal that is routed to antenna system 270 and may pass through a power amplifier (not shown). The power amplifier amplifies the RF transmit signal and routes it to antenna system 270, where the signal is switched to the antenna port for transmission.

Baseband system 260 is also communicatively coupled with processor 210, which may be a central processing unit (CPU). Processor 210 has access to data storage areas 215 and 220. Processor 210 is preferably configured to execute instructions (i.e., computer programs, such as the disclosed application, or software modules) that can be stored in main memory 215 or secondary memory 220. Computer programs can also be received from baseband processor 260 and stored in main memory 210 or in secondary memory 220, or executed upon receipt. Such computer programs, when executed, enable system 200 to perform the various functions of the disclosed embodiments.

Embodiments of systems and methods for a personal diagnostic platform will now be described in detail. It should be understood that the described processes may be embodied in one or more software modules that are executed by one or more hardware processors (e.g., processor 210), for example, as the application discussed herein (e.g., server application 112, client application 132, and/or a distributed application comprising both server application 112 and client application 132), which may be executed wholly by processor(s) of platform 110, wholly by processor(s) of user system(s) 130, or may be distributed across platform 110 and user system(s) 130, such that some portions or modules of the application are executed by platform 110 and other portions or modules of the application are executed by user system(s) 130. The described processes may be implemented as instructions represented in source code, object code, and/or machine code. These instructions may be executed directly by hardware processor(s) 210, or alternatively, may be executed by a virtual machine operating between the object code and hardware processors 210. In addition, the disclosed application may be built upon or interfaced with one or more existing systems.

Alternatively, the described processes may be implemented as a hardware component (e.g., general-purpose processor, integrated circuit (IC), application-specific integrated circuit (ASIC), digital signal processor (DSP), field-programmable gate array (FPGA) or other programmable logic device, discrete gate or transistor logic, etc.), combination of hardware components, or combination of hardware and software components. To clearly illustrate the interchangeability of hardware and software, various illustrative components, blocks, modules, circuits, and steps are described herein generally in terms of their functionality. Whether such functionality is implemented as hardware or software depends upon the particular application and design constraints imposed on the overall system. Skilled persons can implement the described functionality in varying ways for each particular application, but such implementation decisions should not be interpreted as causing a departure from the scope of the invention. In addition, the grouping of functions within a component, block, module, circuit, or step is for ease of description. Specific functions or steps can be moved from one component, block, module, circuit, or step to another without departing from the invention.

Furthermore, while the processes, described herein, are illustrated with a certain arrangement and ordering of subprocesses, each process may be implemented with fewer, more, or different subprocesses and a different arrangement and/or ordering of subprocesses. In addition, it should be understood that any subprocess, which does not depend on the completion of another subprocess, may be executed before, after, or in parallel with that other independent subprocess, even if the subprocesses are described or illustrated in a particular order.

The systems and methods described below consist of several different platforms, processes, and hardware components working together to provide a comprehensive personal diagnostic platform that also allows for robust contact tracing. These components include a smart diagnostic platform, a personal diagnostic device that can interface with the platform to provide medical data, and a guest tracking system that will track guests and that can be used to monitor capacity limits, enforce distancing protocols and perform contact tracing when needed.

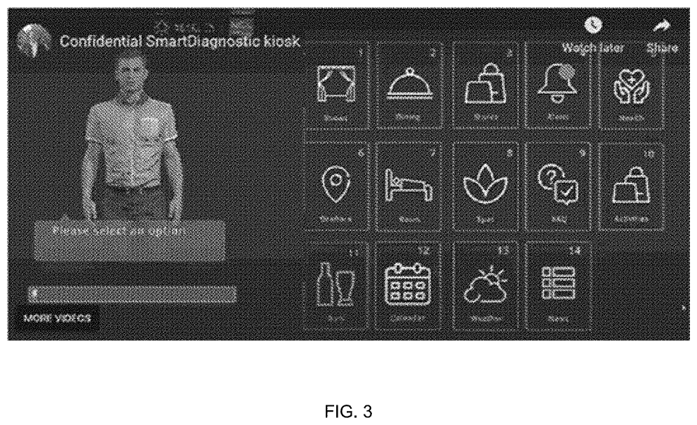

FIG. 3 is a screen shot illustrating a user interface for the personal diagnostic platform according to one embodiment. As can be seen in the example of FIG. 3, a type of concierge service can be incorporated into the platform that can assist with such things as dining reservation, port excursion booking, and ship activity schedule. Of course, this is for a cruise ship example, but obviously the assistance offered through the interface and platform can correspond to different services offered by different entities or companies. Thus, the platform can be deployed on a cruise ship, in an office building(s), a school, in-home, or elsewhere.

The user interface can also have a customizable avatar that can act as a virtual butler for each cabin or room. The interface can be motion and/or voice activated. Thus, the user can just speak commands and requests and not only will the user interface display different information, but the avatar can respond with voice feedback as well. The user interface can also incorporate multi-language capability and facial recognition.

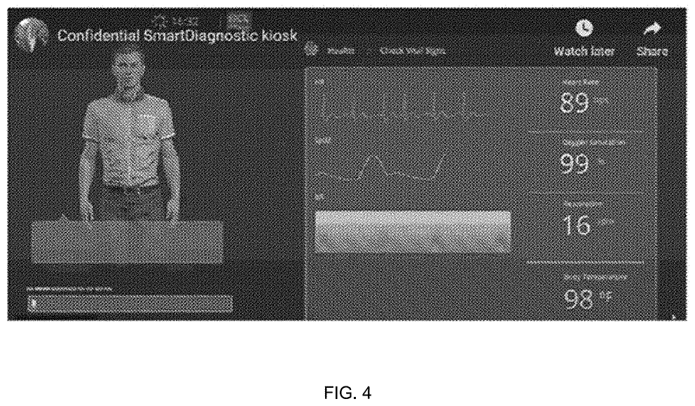

With respect to the health of the passenger, the user interface can be used to track health data such as temperature, heart rate, pulse oximetry, and lung condition/activity. The user interface can also be used to triage situations where the passenger feels they are not well. For example, rather than going to a clinic on the ship, or in an office complex, the user can activate the healthcare functionality and the user interface can obtain information as to the passenger's condition. This can be via voice interaction or responses to a series of questions present in the user interface. Also, as explained below, a diagnostic device or devices can interface with the platform in order to measure and track temperature, heart rate, pulse oximetry, lung condition/activity, etc., as illustrated in FIG. 4.

All of this information can then be used, e.g., by a medical professional, to determine what action should be taken. Note that the medical professional does not necessarily need to be on the ship, or generally at the same location.

The diagnostic platform can comprise a smart diagnostic telemedicine AI module that can also assist with diagnosis or suggest course of action. The diagnostic platform can also maintain a comprehensive passenger health report for destination port access prior to offboarding, increase medical staff safety by reducing in-person interactions with isolated passengers, measure treatments effectiveness using live data and historical data, and onboard historical data tracking provides actionable health information for medical staff.

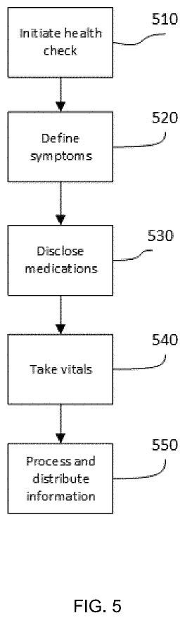

FIG. 5 illustrates an example of at least a portion of a series of questions that can be asked via the user interface in order to illicit information and data to use by the telemedicine module, or a healthcare professional. Sensor data can be histological as well as graphical. The interface can provide an avatar with which the user can interact. Interactions can be through various means, including voice command, touchscreen, and/or remote input device. In a first interaction 510, a user can instruct the diagnostic platform to initiate a check of the user's health. In a second interaction 520, the user can input any symptoms the user may be experiencing (e.g., shortness of breath). In a third interaction 530, the user can notify the diagnostic platform of any medications that the user is currently taking. In a fourth interaction 540, the user's vital measurements are taken by a diagnostic device. In one embodiment of the present invention, the vital measurements include temperature, heart rate, and pulse oximetry. In a fifth interaction 550, the information is processed on a back-end server and delivered to the appropriate medical provider. The user interface can be integrated into the passenger's television. Alternatively, a tablet or other dedicated device can be provided in each cabin for accessing the user interface. A camera can be interfaced with, or integrated in the display to allow observation of the passenger or to videoconference with the passenger, either during the input of healthcare information, or afterwards, e.g., as a follow up. But it should be noted that the avatar is intended to handle most of the interaction and guidance of the passenger in order to relieve the healthcare professional.

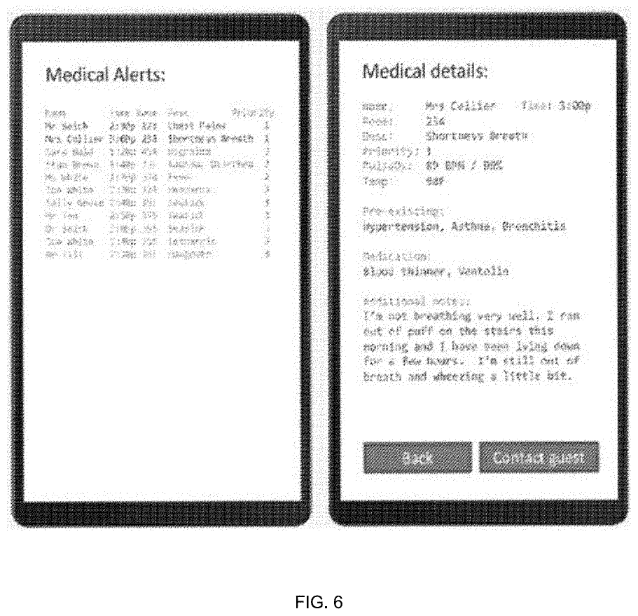

FIG. 6 illustrates an example user interface for medical staff. As can be seen, medical alerts can be logged and details about specific alerts/patients can be accessed.

In certain embodiments, the user interface can be an application that is downloaded to a smartphone, tablet, etc. As noted below, the sensors included in the diagnostic device can be included in the device to which the app. is downloaded. In other words, the device can include or comprise the diagnostic device described below. The diagnostic device can also be incorporated into a watch or other wearable device.

In fact, in certain embodiments, the diagnostic platform does not need a display due to the two-way verbal interaction with the avatar functions. In other words, the avatar can walk the user through the process. As such the platform can be put into almost any form factor such as a medicine cabinet door, a lamp, a clock radio, a smart speaker, etc.

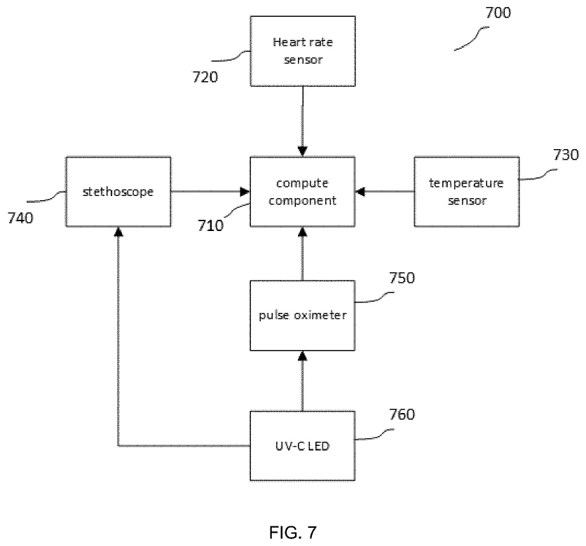

FIG. 7 is a block diagram of the sensor array of an example diagnostic device 700 that can be used in conjunction with the diagnostic platform according to one embodiment. The diagnostic device can have several sensors, capabilities and can be used to track temperature, heart rate, pulse oximetry, EKG, and can be used as a stethoscope. Shown in FIG. 7 is a compute component 710 which can receive information from sensors. Temperature of a user can be obtained through a temperature sensor or thermometer 730, heart function information through heart rate sensor or EKG 720, lung function through a stethoscope 740, and the proportion of oxygenated hemoglobin in the blood through a pulse oximeter 750. In one embodiment of the invention, the diagnostic device also includes UV-C light emitting diodes 760 which are capable of sterilizing the pulse oximeter 750, or other surfaces that come in contact with the user, such as the stethoscope 740. Moreover, the device or a docking station/cradle can include UV sterilization capability to sterilize the contact surfaces of the diagnostic device.

The device can be placed on chest to operate as a digital stethoscope to provide data on heart and breathing sounds. A finger can be placed on top of a sensor to obtain accurate read of pulse and blood oxygen levels.

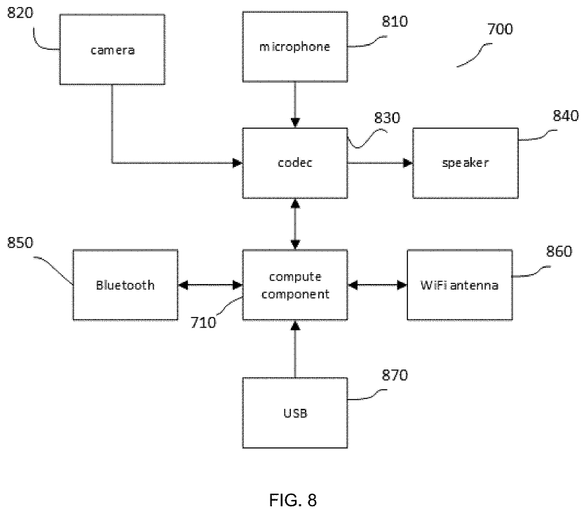

FIG. 8 is a block diagram of the input/output capabilities of an example diagnostic device 700 that can be used in conjunction with the diagnostic platform according to one embodiment. Shown in FIG. 8 is a compute component 710 which can receive information from devices, such as a microphone 810 and a camera 820, which in one embodiment is mediated by a codec 830 so that information can be processed by the compute component 710. The compute component may also provide feedback through the speaker 840. Additionally, the device can include a USB 870 or other type of connector to allow for charging and for communication with the display device. Alternatively, or additionally, the device can have short range wireless communication capability such as Bluetooth 850 or WiFi 860.

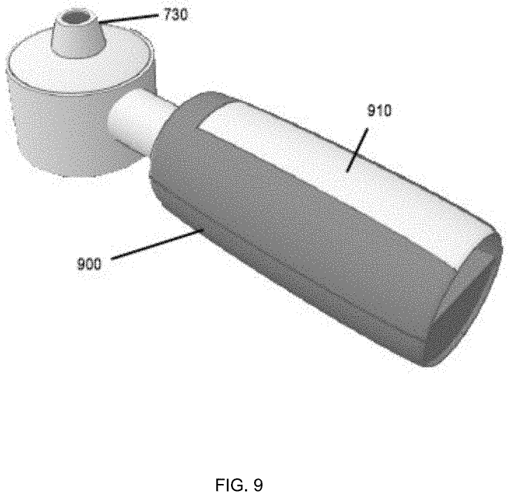

FIG. 9 illustrates another example embodiment of a diagnostic device that can, e.g., be used with the diagnostic platform described herein. As can be seen, the device can include a contactless temperature sensor 730. The handle 900 can also include a pressure clip 910 that the user can slide their finger under to contact the pulse oximeter sensor.

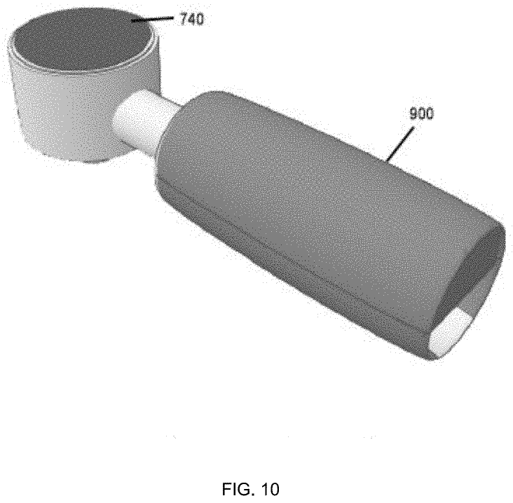

FIG. 10 illustrates the flip side of the diagnostic device in which can be seen the handle 900 and a digital stethoscope 740.

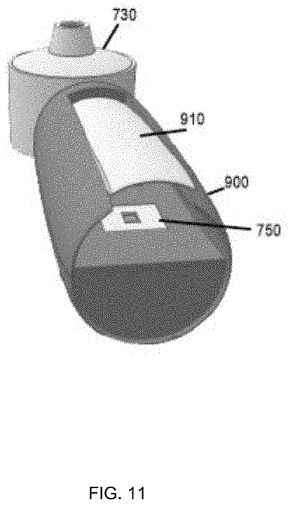

FIG. 11 illustrates a bottom oblique view of the diagnostic device in which can be seen the temperature sensor 730, handle 900, pressure clip 910, and under the pressure clip 910 is pulse oximetry sensor 750. In one embodiment of the present invention, the handle 900 can include a pressure clip 910 that the user can slide their finger under to contact the pulse oximeter sensor. In one embodiment of the present invention, a UV sensor can be included on the underside of the clip to, e.g., sterilize the pulse oximeter sensor 750.

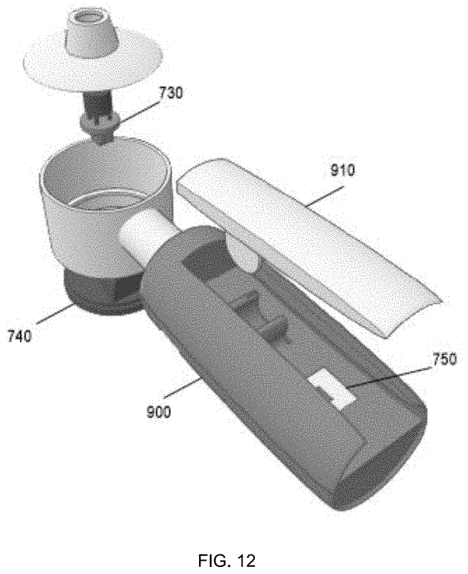

FIG. 12 illustrates a side oblique exploded view of the diagnostic device in which can be seen the temperature sensor 730, handle 900, pressure clip 910, and under the pressure clip 910, and the pulse oximeter sensor 750.

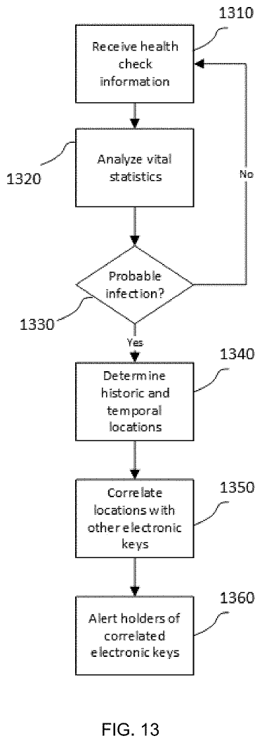

FIG. 13 is a flow chart that illustrates a process that the diagnostic platform can perform in an embodiment of the present invention. When a health check is initiated, the information obtained from the interview or interaction with the avatar and the vital statistics obtained from a diagnostic device are received by the back-end server 1310. The back-end server can then analyze the received health care information 1320 and determine whether the user is likely infected with a transmissible disease or virus 1330. If the user is does not have a threshold probability of infection, the diagnostic platform will wait to receive information from another health check 1310. If the user does have a threshold probability of infection, the back-end server will determine the historic temporal and physical locations of the user within the environment during the probable contagion period 1340. In one embodiment of the present invention, the environment can be any demarcated area that is surveilled by electronic keys and electronic key holders, such as a cruise ship, office building, or event center. The back-end server will have data on the users locations by communicatively receiving data from the electronic key readers as they are activated by the user's electronic key. The back-end server will then correlate the historic temporal and physical locations of the infected user with the temporal and physical locations of the other electronic keys 1350. Users who hold electronic keys whose locations are correlated with the infected user's location will be alerted 1360.

In an environment like a ship or a building or campus, contact tracing information can also be gathered and used in conjunction with the health data gathered by the platform described above. For example, time stamped data can be gathered from user key cards, or other cards or devices, to completely automate contact tracing, to measure space occupancy and to provide tracking data for venues.

Readers can be set up at key points and configured to receive data from the key card or other device. In this way, the guest's location can be tracked. That data can then be combined with the data for other guests and can be used to trace potential contact between guests if such contact tracing is deemed necessary, e.g., based on the health data gathered via the diagnostic platform. This can cause the diagnostic platform to inform potential contacts via the user interface and to request certain information and vital measurements from those potential contacts. Alternatively, the potential contacts can be requested to go to a healthcare location, e.g., clinic within a ship, to be tested, or personal can be dispatched to the, e.g., rooms of potential contacts.

Readers are generally known commodities. Readers integrate with RFID tags, wristbands, smartphone apps, and other wearable devices. Interaction and layering of RFID tracking can be integrated into the software hosted by the diagnostic platform. For example, if a user has a health check-in and shows a fever, the external RFID tracking system can identify that the user was in a specific location at a specific time, and can associate with others who were at that location at that time. The RFID software can determine, based on digital blueprints of the environment, where people are located. The diagnostic platform can capture when there is an alert, and that contact information can be layered as another widget on the diagnostic platform.

Longer range wireless communication technologies, such as UHF RFID can be used as well. With this type of technology, a single reader can detect user's cards within large areas. This type of technology can be used not only for tracing but to enforce distancing and capacity limitations. For example, the system will know how many people are in a particular room or venue and can indicate, e.g., via red or green light, whether other people are free to enter or must wait. Bluetooth can also be used so that phones, tables, watches, customized key cards, etc., can be used as well.

It will also be understood that combinations of wireless technology can also be use, e.g., a shorter range technology can be combined with a larger range technology for tracing at different locations and in different environments. In one embodiment of the present invention, a keycard includes both NFC, for close range, and ISO16000-6C, for long range, capabilities.

While certain embodiments have been described above, it will be understood that the embodiments described are by way of example only. Accordingly, the systems and methods described herein should not be limited based on the described embodiments. Rather, the systems and methods described herein should only be limited in light of the claims that follow when taken in conjunction with the above description and accompanying drawings.

It should be further understood that the examples and embodiments pertaining to the systems and methods disclosed herein are not meant to limit the possible implementations of the present technology. Further, although the subject matter has been described in a language specific to structural features and/or methodological acts, it is to be understood that the subject matter defined in the appended claims is not necessarily limited to the specific features or acts described above. Rather, the specific features and acts described above are disclosed as example forms of implementing the Claims.

Since other modifications and changes varied to fit particular operating requirements and environments will be apparent to those skilled in the art, the invention is not considered limited to the example chosen for purposes of disclosure, and covers all changes and modifications which do not constitute departures from the true spirit and scope of this invention.

Since other modifications and changes varied to fit particular operating requirements and environments will be apparent to those skilled in the art, the invention is not considered limited to the example chosen for purposes of disclosure, and covers all changes and modifications which do not constitute departures from the true spirit and scope of this invention.

Since other modifications and changes varied to fit particular operating requirements and environments will be apparent to those skilled in the art, the invention is not considered limited to the example chosen for purposes of disclosure, and covers all changes and modifications which do not constitute departures from the true spirit and scope of this invention.

What is claimed is:

1. A diagnostic platform, comprising:
a database;
a display, and
a user interface displayed via the display, the user interface comprising:
a plurality of screens configured to provide information and allow or prompt the input of health information;
an avatar capable of two-way verbal communication that can provide information and help navigate through the screens;
a back-end server configured to receive the health information, make the health information available to healthcare personnel, and store the healthcare information.

2. The diagnostic platform of claim 1, wherein the display is an in-room unit.

3. The diagnostic platform of claim 1, wherein the display is incorporated into a device selected from the group consisting of a tablet, a smartphone, a television, and a computer.

4. The diagnostic platform of claim 1, further comprising:
a plurality of electronic key readers;
a plurality of electronic keys;
wherein said plurality of electronic key readers are configured to communicate with said plurality of electronic keys, and said plurality of electronic key readers are configured to communicate to said back-end server to transmit information related to the location of said electronic keys, whereby said back-end server can calculate the historical and current proximity of each of the said plurality of electronic keys to each other.

5. The diagnostic platform of claim 1, further comprising a diagnostic device configured to communicate with said back-end server, said diagnostic device comprising:
a handle;
a temperature sensor;
a heart rate sensor;
a pulse oximeter sensor; and
a stethoscope.

6. The diagnostic platform of claim 5, wherein said diagnostic device further comprises a camera.

7. The diagnostic platform of claim 5, wherein said diagnostic device further comprises a microphone and a speaker.

8. The diagnostic platform of claim 5, wherein said diagnostic device further comprises a UV-C light emitting diode configured to provide ultra violet sterilization of said pulse oximeter sensor.

9. The diagnostic platform of claim 5, wherein said diagnostic device is incorporated into a device selected from the group consisting of a tablet, a smartphone, and a wearable device.

10. The diagnostic platform of claim 5, wherein said diagnostic device further comprises a wireless communication interface.

11. A diagnostic device, said diagnostic device comprising:
a handle;
a temperature sensor;
a heart rate sensor;
a pulse oximeter sensor; and
a stethoscope.

12. The diagnostic device of claim 11, further comprising a camera.

13. The diagnostic device of claim 11, further comprising a microphone and a speaker.

14. The diagnostic device of claim 11, further comprising a a UV-C light emitting diode configured to provide ultra violet sterilization of said pulse oximeter sensor.

15. The diagnostic device of claim 11, wherein said diagnostic device is incorporated into a device selected from the group consisting of a tablet, a smartphone, and a wearable device.

16. The diagnostic device of claim 11, further comprising a wireless communication interface.

17. A computing system comprising a non-transitory computer-readable medium with an executable program stored thereon, wherein the program instructs a processor to perform operations comprising:
receiving temporal and physical location data from a plurality of electronic key readers communicating with a plurality of electronic keys;
receiving healthcare information from a user, wherein said user is a holder of one of said plurality of electronic keys;
analyzing said healthcare information to determine whether said user has a transmissable infection;
determining the historic temporal and physical locations of said user utilizing said location data;
correlating historic temporal and physical locations of said user's electronic key with historic temporal and physical locations with each of the other plurality of electonic keys; and
alerting holders of each of the other plurality of electronic keys that are correlated with said user's electronic key.

18. The computing system of claim 17, wherein receiving said healthcare information includes information obtained from a user from a display, and a user interface displayed via the display, the user interface comprising:
a plurality of screens configured to provide information and allow or prompt the input of health information; and
an avatar capable of two-way verbal communication that can provide information and help navigate through the screens.

19. The computing system of claim 17, wherein receiving said healthcare information includes information obtained from a diagnostic device comprising:
a handle;
a temperature sensor;
a heart rate sensor;
a pulse oximeter sensor; and
a stethoscope.

20. The computing system of claim 17, wherein said healthcare information includes temperature, heart rate, pulse oximeter reading and lung function.
